(19) United States
(12) Patent Application Publication
(10) Pub. No.: US 2024/0188915 A1
(43) Pub. Date: Jun. 13, 2024

(54) NORMAL PRESSURE HYDROCEPHALUS TREATMENT AND ANALYSIS

(71) Applicants: Sharada Kadaba Sridhar, Minneapolis, MN (US); Rui Kuang, Plymouth, MN (US); Uzma Samadani, Wayzata, MN (US)

(72) Inventors: Sharada Kadaba Sridhar, Minneapolis, MN (US); Rui Kuang, Plymouth, MN (US); Uzma Samadani, Wayzata, MN (US)

(21) Appl. No.: 18/534,822

(22) Filed: Dec. 11, 2023

Related U.S. Application Data
(60) Provisional application No. 63/431,539, filed on Dec. 9, 2022.

(51) Int. Cl. A61B 6/50; A61B 6/00; G06T 7/00; G06T 7/11

(52) U.S. Cl. CPC A61B 6/501; A61B 6/5217; A61B 6/5258; G06T 7/0012; G06T 7/11; G06T 2207/10081; G06T 2207/30016

(57) ABSTRACT

A method includes treating a patient diagnosed by a process, the method including determining, based on a representation of a first tissue, a region of interest of the patient, and determining, based on the region of interest, an estimate of a location of a commissure within a representation of second tissue of the patient.

### CROSS-REFERENCE TO RELATED APPLICATIONS

The present application claims priority to U.S. Provisional Ser. No. 63/431,539, filed Dec. 9, 2022, the content of which is hereby incorporated by reference in its entirety as if fully set forth herein.

### BACKGROUND

There is a vital need for large-scale screening and differentiation of idiopathic Normal Pressure Hydrocephalus (NPH) from Alzheimer's Dementia (AD) and Posttraumatic Encephalomalacia (PTE), with early intervention leading to better outcomes.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a computer (e.g., a special purpose computer), or other programmable data processing apparatus to produce a machine, such that the processor-executable instructions which execute on the computer or other programmable data processing apparatus create a device for implementing the functions specified in the flowchart block or blocks.

This detailed description may refer to a given entity performing some action. It should be understood that this language may in some cases mean that a system (e.g., a computer) owned and/or controlled by the given entity is actually performing the action.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a special purpose computer or other programmable data processing instrument to produce a machine, such that the processor-executable instructions which execute on the computer or other programmable data processing instrument create a device for implementing the steps specified in the flowchart block or blocks.

These processor-executable instructions may also be stored in a non-transitory computer-readable memory or a computer-readable medium that may direct a computer or other programmable data processing instrument to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing instrument to cause a series of operational steps to be performed on the computer or other programmable instrument to produce a computer-implemented process such that the processor-executable instructions that execute on the computer or other programmable instrument provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

The method steps recited throughout this disclosure may be combined, omitted, rearranged, or otherwise reorganized with any of the figures presented herein and are not intend to be limited to the four corners of each sheet presented.

The techniques disclosed herein may be implemented on a computing device in a way that improves the efficiency of its operation. As an example, the methods, instructions, and steps disclosed herein may improve the functioning of a computing device.

Radiographic features that capture ventricular morphology in relation to brain atrophy may enable objective classification of Normal Pressure Hydrocephalus (NPH) from Alzheimer's Dementia (AD), Post-traumatic Encephalomalacia (PTE), and headache controls (HC) that is accurate, sensitive, and free from inter- and intra-observer variability. Our goal is to develop computational pipelines for automatic extraction of Non-Contrast Computed Tomography (NCCT) features of ventriculomegaly, and to train a deep regression network to predict landmarks for image standardization.

A retrospective cohort is pulled from the Veterans Affairs database VINCI, and is classified into NPH, AD, PTE, or HC. Image processing pipelines are developed to extract the Evans indices (x, y, z), Callosal Angle (CA), a proxy-Splenial Angle (p-SA), Normalized Maximum Third Ventricle Width (NMax-3VW), and CSF to brain volume ratio (CSF2BVR). A novel feature that captures lateral ventricle enlargement (MaxEccLV) is also introduced. A deep regression network is trained to predict the anterior and posterior commissure (AC, PC) landmarks which are required for image standardization. Group differences in the features are examined using t-tests for means. Random Forest feature importance is used for feature selection, followed by logistic regression for classification of NPH, AD, PTE, and HC.

A subset of patient scans with Post-Traumatic chronic Subdural Hemorrhage (PT-cSDH) are used to report PTE characteristics. On 115, 169, 64, and 80 NCCT scans of 89 NPH, 108 AD, 64 PT-cSDH, and 79 HC patients respectively, the measurements of EI-x, EI-y, EI-z, CA, MaxEccLV, p-SA, NMax-TVW, and CSF2BVR were successful in 95.5%, 99%, 99%, 92.7%, 95.7%, 90.3%, 98.8%, 100% of cases. Significant differences were present in MaxEccLV (95% CIs: [0.12,0.14], [0.07,0.1], [0.08,0.12]), EI-x ([−0.14,−0.12], [−0.05,−0.03], [−0.08,−0.05]), EI-y ([−0.05,−0.03], [−0.03,−0.01], [−0.03,−0.01]), EI-z ([−0.21,−0.18], [−0.11,−0.08], [−0.14,−0.1]), CA ([19.09,31.41], [18.51,29.25], [20.05,32.89]), p-SA ([6.06, 16.68], [3.95, 10.96], [9.94,21.19]), NMax3VW ([−0.07,−0.05], [−0.03,−0.02], [−0.04,−0.03]), and CSF2BVR ([−0.1,−0.08], [−0.05,−0.03], [−0.07,−0.05]) between NPH and the HC, AD, PT-cSDH cohorts respectively. Considering cases where all features were successfully computed, repeated 5-fold cross validation using logistic regression results in a sensitivity and specificity of 87% and 84% for NPH v/s AD, 97% and 98% for NPH v/s HC, 91% and 84% for NPH v/s PTcSDH, and 85.71% and 89.42% for NPH v/s AD/PT-cSDH/HC respectively.

Conclusions: The computational pipelines for the algorithmic extraction of ventriculomegaly features from NCCT were successful in more than 90% of the scans, each. The novel MaxEccLV suggests that “rounder” ventricles are more suggestive of hydrocephalic pathology, and significantly improved the distinction between NPH and AD. The sensitivity and accuracy of NPH classification suggest that these features can be used to screen for suspected NPH and distinguish it from AD and PT-cSDH. Unlike a prior work that algorithmically extracts EI-x from NCCT, our contribution does not depend on nonlinear registration for image standardization which introduces brain deformation that may affect comparative analysis. We use a linear transformation based on the location of AC and PC landmarks predicted by a deep regression model.

There is a vital need for large-scale screening and differentiation of idiopathic Normal Pressure Hydrocephalus (NPH) from Alzheimer's Dementia (AD) and Posttraumatic Encephalomalacia (PTE), as it is thought to be a prominent type of reversible dementia that may be treated with shunt-surgery, with early intervention leading to better outcomes. NPH patients received inaccurate diagnosis with most cases attributed to AD or Parkinson's Disease (PD), due to overlapping clinical symptoms like gait impairment and dementia. Post-traumatic encephalomalacia (PTE) refers to atrophy or softening of brain tissue that occurs after head injury. Ventriculomegaly in NPH due to hydrocephalus, and its apparent presence in AD and PTE due to atrophy is challenging to distinguish by visual inspection of radiographic scans, which underscores the need for algorithmic evaluation and differentiation. MRI is preferentially utilized for radiographic evaluation due to their higher soft-tissue resolution, despite their many contraindications, higher cost, and longer duration.

Highly sensitive methods for automatic Non-Contrast Computed Tomography (NCCT) based detection of NPH have not been developed. Nonlinear registration for standardization may affect measurements as the width of the inner cranium is relatively constant after registration. A quantification of Disproportionately Enlarged

Subarachnoid-space Hydrocephalus (DESH) on NCCT scans using the SILVER index defined as the ratio of the sylvian fissure area to the subarachnoid space area at the vertex may distinguish NPH from controls. While final clinical diagnosis should rely on examining multi-modal data and symptomatic presentations, noninvasive screening using NCCT scans that are frequently acquired in emergency settings may increase cases where patients with reversible dementia due to NPH, are correctly identified and treated.

Image standardization is crucial for comparative analysis, and the Anterior Commissure (AC) and Posterior Commissure (PC) may be used. Knowledge of actual locations of these landmarks are desirable as they are close to important landmarks such as the foramen of Monroe and the cerebral aqueduct, especially on NCCT due to their lower resolution. They also help with uniquely determining the midline which is necessary to correct for patient head tilt during scans. We pose the prediction of AC and PC landmarks on NCCT scans as a heatmap regression problem for fine localization that follows a prior anatomical knowledge-based method for coarse localization. A 3D UNet for regression is trained to minimize a modified cross entropy loss to handle imbalance in class distribution.

We develop automatic computational methods to extract NCCT features of ventriculomegaly using image processing pipelines. Such a characterization of ventriculomegaly in NPH, AD, PTE, and headache controls (HC) may result in proper identification and treatment of individuals with NPH. As the nature of injury can be highly diverse in PTE, we report results on a subset of patients with post traumatic chronic subdural hematomas (PT-cSDH). Firstly, semi-automated computational pipelines are developed using image processing to extract the following radiographic features from NCCT: Evans Indexx (EI-x), Evans Indexy (EI-y), Evans Indexz (EI-2), Callosal Angle (CA), proxy-Splenial Angle (p-SA), Normalized Maximum Third Ventricle Width (NMax-3VW), and CSF to brain volume ratio (CSF2BVR). A novel feature computed as the maximum Eccentricity of best fitting ellipses to a standard coronal view of the left and right Lateral Ventricles (MaxEccLV) is also extracted. These features are expected to capture the effects of ventriculomegaly on the brain. Next, a 3Dpatch based U-Net model is developed to predict the 3D coordinates of standard landmarks (AC and PC), posed as a voxel-wise heatmap regression problem. This enables the prediction of landmarks required for image standardization and complete automation of ventriculomegaly feature extraction, allowing large-scale assessment for screening and classification of NPH.

With Institutional Review Board approval and a waiver for informed consent, all patients who presented for an NCCT scan at the VA from 2000-2019 were identified along with their diagnoses, subsequent scans, age, and gender on the VA Networking and Communication Infrastructure (VINCI) system. Death was not an exclusionary factor. To ensure the validity of NPH, AD, and PTE (and PT-cSDH) diagnoses primarily identified through International Classification of Disease (ICD) codes, the clinical and radiographic reports associated with the patients were also reviewed. To avoid selection bias, all patients diagnosed with NPH whether it is possible, probable, or confirmed irrespective of their tap-test or shunt-response were included. It was also ensured that the NPH, AD, and PTE cohorts were mutually exclusive along with no history of Vascular Dementia (VaD), Frontotemporal dementia (FTD), Dementia with Lewy Bodies (DLB), Parkinson's Disease (PD), tumor, meningitis, or stroke. A cohort was pulled as headache control (HC) which included patients who presented to the VA for a headache and had a negative head CT, with no mention of dementia or head trauma. Their NCCT scans were downloaded from the VA radiology picture archiving and communication system. All patient identifiers from DICOM images were then stripped or randomized, according to a pre-approved VA protocol and transferred to a computer for further analysis.

All scans were acquired on Philips, Siemens, or Toshiba scanners. Most scans were acquired with a peak tube voltage of 120 kVp, with a few acquired at 100 kVp and 130 kVp. The gantry/detector tilts varied from −21 to +15 degrees, slice thickness from 1 mm to 5 mm, and X-ray tube current from 62 to 500 mAs. The median number of slices was 32 and the median pixel spacing was 0.44 mm×0.44 mm.

The preprocessing steps are gantry tilt correction, isotropic resampling, bed removal, and skull stripping, in that order. NCCT images are often acquired with a gantry tilt to minimize exposure to sensitive areas such as the eyes. If this effect is not corrected, the image might appear skewed and affect further analysis. 3D Slicer's DICOM importer module is used to address this issue and correct for irregular slice thickness or any missing slices. Isotropic resampling is then performed to ensure that all voxels are of size 1 mm×1 mm×1 mm, followed by bed removal. The skull is stripped in a slice-by-slice fashion. It involves gaussian filtering, erosion, largest component retention, dilation, and morphological closing to fill any holes. The brain is extracted for feature extraction. Image orientation can be standardized before measuring any comparative analysis. We may perform linear transformation to retain the relative sizes of different brain anatomies. The AC, PC, and midline (for patient head tilt correction) may be manually annotated on all scans. A linear transformation is then applied to resample the images to make the line connecting the AC and PC horizontal and the interhemispheric fissure vertical. A 3D slicer may be used for all preprocessing. The deep regression model defined subsequently learns to predict the AC and PC locations to replace the manual annotation step. The mid sagittal plane (MSP) is uniquely defined by the AC, PC, the centroid of the brain, and the points sampled on the ventricular midline for patient head tilt correction.

Ventricle segmentation is carried out before computation of the radiographic features. Intensity-based segmentation and the use of morphological operations may be used to clean up any non-ventricular tissue. Brain images may be windowed from 0-100 HU and smoothed using a 3×3 Gaussian kernel for denoising. The images may then be binarized using the Otsu threshold method. As the ventricles are near 0 HU, they are segmented from the brain parenchyma.

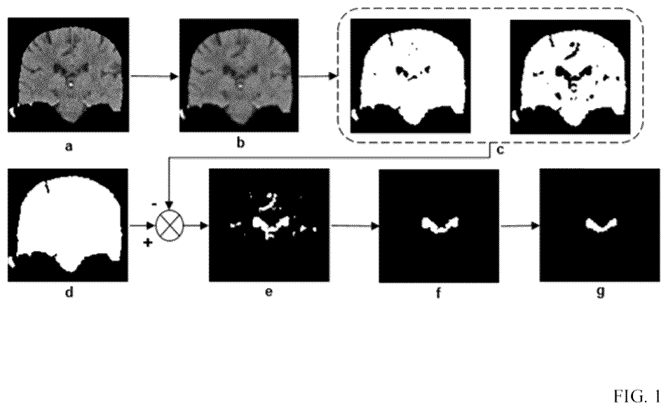

There are some cases where Otsu thresholding alone will be unable to accurately capture a crude ventricle mask, potentially due to high CSF protein content (HU value), periventricular hypodensity, or very small sized ventricles. Adaptive intensity thresholding with a gaussian kernel (size of 75 mm as informed by preliminary experiments) is used to address this problem and provide better segmentation as it is a local approach. Information from the global and local segmentation approaches are combined to produce an overall brain mask. Flood-filling is performed on this mask to extract an intracranial space mask. The mask is then subtracted from the intracranial space mask which results in a primary segmentation of the ventricles. Presence of choroid plexus, prominent midline, atrophy, and dilated sulci in the proximity of the ventricles might affect the segmentation. Removal of non-ventricle tissue is handled separately for each computational pipeline using specific morphological operations and domain knowledge. The segmentation process is illustrated in FIG. 1.

Canny edge detection is then used to find the edges of the segmented ventricles, followed by Moore contour tracing (MCT). Starting at an initial point, the algorithm seeks to find the boundary points by imitating a bug that traverses the plane as follows. When it hits a foreground pixel (f), it records the location, backtracks, and traverses the moore neighborhood (8 surrounding pixels) in a fixed direction (clockwise) until it hits another foreground pixel. It repeats this process of hitting and collecting foreground pixels until the initial point is revisited.

The Splenial Angle (SA) is a measure which is correlated to the compression of the white matter tracts of the corpus callosum known as the forceps major, due to ventricular distention. It was originally measured on a standard axial plane and on Diffusion Tensor Imaging (DTI) Fractional Anisotropy maps.

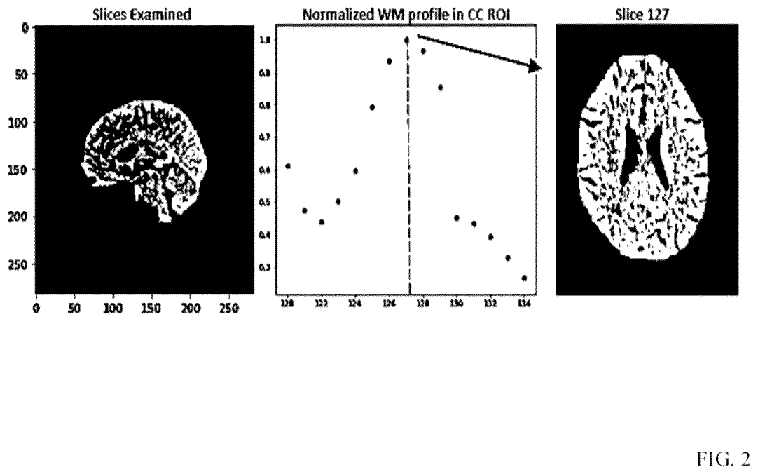

White Matter (WM) is poorly imaged on NCCT. We define a proxy measure, the p-SA, which is defined as the angle between the medial walls of the occipital horns of the lateral ventricles as imaged on the first axial plane with the body of the corpus callosum (CC) completely visible while scrolling in the inferior-superior direction. The definition is based on the observation that the forceps major might follow the medial walls of the occipital horns steadily and that the ventricles are easier to segment on NCCT than WM. As shown in FIG. 2, the p-SA computational pipeline begins by locating the point 5 mms below the superior most and middle point of the lateral ventricles on the MSP. This is achieved by first segmenting the lateral ventricles on a sagittal reconstruction. WM is segmented from axial images using a simple intensity thresholding of 22 to 28 HU, and a Corpus Callosum (CC) region of interest (ROI) is defined to best capture the body visible between the lateral ventricles. Axial slices 15 mm above the identified starting point are used to plot a normalized white matter profile. The point at which the signal reaches a maximum value of 1 is picked as the standard axial plane for p-SA measurement. The axial plane is then used to segment the lateral ventricles. The contours of the medial walls of the left and right occipital horns are traced and limited by the centroid and the inferior most points in the south-west and south-east directions respectively. The angle between the best fitting lines to these contours is computed to calculate p-SA.

Based on the observation that the lateral ventricles of patients who suffered from NPH had a “rounder” appearance on the standard coronal plane at the level of the PC, we hypothesized that the maximum eccentricity of ellipses that approximate the left and right lateral ventricles (LLV and RLV) would be smaller in NPH patients as opposed to AD patients. A computational pipeline to trace the contour of the segmented LVs from the centroid of the image to the leftmost and rightmost points at the level of the centroid height is developed. These contour points are then flipped in the x and y directions around the centroids of the LLV and the RLV to carve a best fitting ellipse to the LVs. This approach is taken as the choroid plexus at the floor of the lateral ventricles might sometimes make the segmentation obscure. The best fitting ellipses' equations are estimated by solving a least squares problem on the traced contour points. The major axis and minor axis of the ellipses are determined to solve for the eccentricity. The maximum of the left and right measurements, defined as MaxEccLV, is computed. This novel feature might add additional value as compared to CA, as it not only measures the angle between the LVs, but their potential impingement onto surrounding WM tracts that might be causing functional deficits.

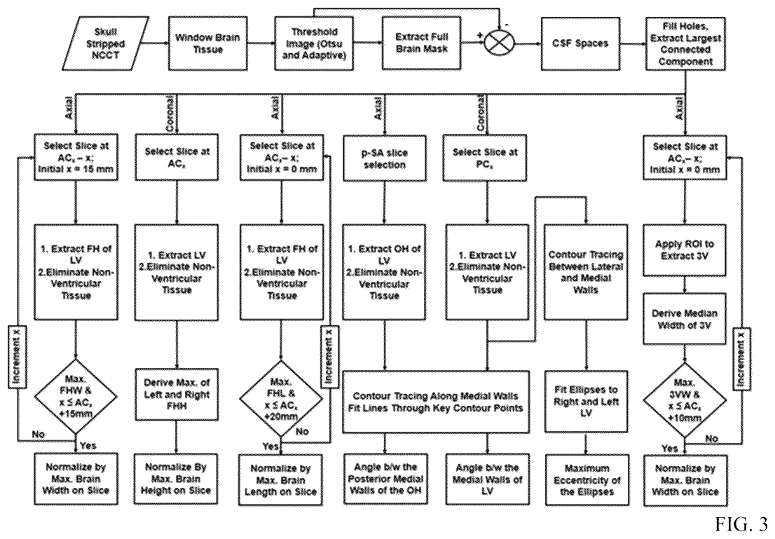

EI-x is a normalized measure of the maximum width of the frontal horns of the lateral ventricles as compared on a range of standardized axial planes, and CA is the angle between the lateral ventricles as measured on the standard coronal plane at the level of the PC. They both capture an enlargement of the lateral ventricles that are not attributable to atrophy. NPH patients are likely to have an EI-x>0.3 and a CA<900 as opposed to AD patients. EI-y is a normalized measure of maximum frontal horn length as compared on a range of standard axial planes, and EI-z measures the normalized frontal horn height on the standard coronal plane at the level of the AC. They both are shown to capture sufficient variation in NPH. The third ventricle is also reported to be enlarged in NPH. We define it as the maximum median width of the third ventricle as compared on a range of standard axial images, normalized by brain width (NMax-3VW). The ratio between CSF to brain volume denoted as CSF2BVR is likely to be higher in NPH than in AD where the brain's volume is truly atrophied. These features are computed based on the ventricle segmentation approach described earlier. The CA, pSA, and MaxEccLV pipelines use MCT to trace key contour points. FIG. 3 illustrates the computational pipelines to extract the radiographic features except for CSFBVR.

Landmark prediction may be framed as a heatmap regression problem using deep neural networks. We approach the task of AC and PC landmark prediction by starting with a coarse localization step that takes prior knowledge into consideration to predict attention ROIs. They then drive the attention of a deep regression model for fine localization. 364 NCCT scans were annotated with the AC and PC for image standardization in the development of the ventriculomegaly feature extraction pipelines. This is used as the ground-truth for AC and PC landmark prediction on isotropic sagittal reconstructions.

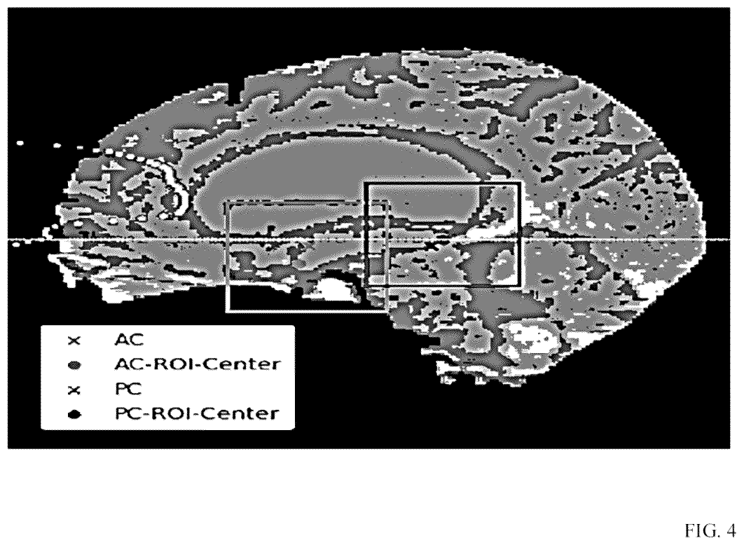

The third ventricle is segmented from the MSP using smoothing, intensity-thresholding, and morphological post-processing steps. As it might appear to be connected to the lateral ventricle in some cases, the column profile of the segmented image is used to detect a separating boundary. In case of significant patient head tilt in the anterior-posterior direction, this method may not work. To address that, the principal component of variation will be identified and made horizontal before evaluating the column profile. The anterior-most and inferior-most point of the third ventricle is chosen as the proposed center (AC-ROI-Center) of the 3D voxel patch for AC prediction, and the posterior-most and inferior-most point as the proposed center (PC-ROI-Center) of the 3D voxel patch for PC prediction (FIG. 4). These landmarks are expected to be reasonably close to the AC and PC (<=15 mm).

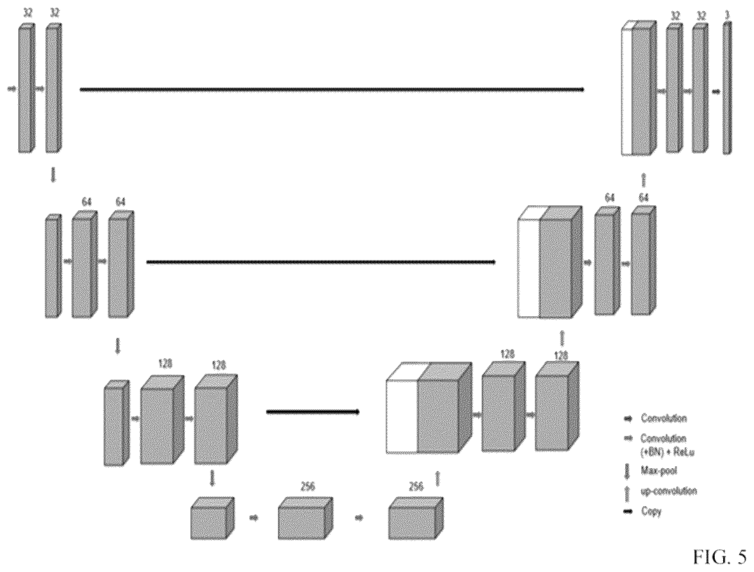

10 3D voxel-patches of dimension 64 mm×64 mm×64 mm (x, y, z) are randomly sampled around AC-ROI-Center and PC-ROI-Center from each scan for training a patch-based 3D U-Net. We use the same number of filters and padded convolutions are used at each level to keep the output size consistent with the input (FIG. 5). Gaussian heat maps are defined on the whole image around the AC (HAC) and PC (HPC) which act as the ground-truth for voxel-wise regression. A background heatmap is defined as 1−(HAC+HPC) to normalize the voxel-wise heatmap distribution, as well as force the model to learn a third background class to address class imbalance. The neural network is trained to regress the 3 heat maps corresponding to the input patch. The network architecture has 4 levels of 2 3×3×3 conv layers followed by ReLU activations in the encoding path with the number of filters increasing from 32 to 256 and a max pooling layer between each. An expansive path that is symmetric to the encoding path where up-convolutions replace max pooling is added. Skip connections from corresponding layers in the encoding path relay contextual information to the expansive path. Batch normalization (BN) is applied before the activation function for faster convergence. A SoftMax layer is applied to each voxel across the 3 channels to obtain voxel probabilities. All layers are initialized with bias 0 and random weights drawn from a Gaussian of variance 0.01. Mini-batch gradient descent is used to train the network by minimizing the loss function:

$$L ( H , \hat{H} ) = - \frac{1}{n} {\sum }_{i = 1}^{n} ( \frac{1}{2} H log \hat{H} + \frac{1}{2} {{α}_{t} ( 1 - {H}_{t} )}^{γ} log {H}_{t} ) ,$$

where the subscript t denotes a specific heatmap, n is the minibatch size, H is the ground-truth heatmap, Ĥ is the predicted heatmap, γ≥0 is the focus parameter, and

$${H}_{t} = \{ \begin{matrix}\hat{H} if H > 0.01 \\ ( 1 - \hat{H} ) otherwise\end{matrix} .$$

This combines the binary cross entropy loss with a focal-loss function which yields higher accuracy in dense object classification. α∈[0,1] and is set as the inverse class frequency and γ is set to 2. It weighs the loss function down when the model confidently predicts a background class that occurs more frequently and amplifies it at less confident predictions of the dense foreground. An initial learning rate of 0.01 is used. The hyperparameters are tuned using 5-fold cross validation. For inference, the prior-knowledge driven module selects 3D voxel patches around AC-ROI-Center and PC-ROI-Center. Overlapping areas are averaged, and the mean locations of the predicted Gaussians in the first two channels after normalization and thresholding are the predicted AC and PC locations. The performance is evaluated using the average Euclidean distance between the predicted and actual landmarks defined as the mean radial error (MRE).

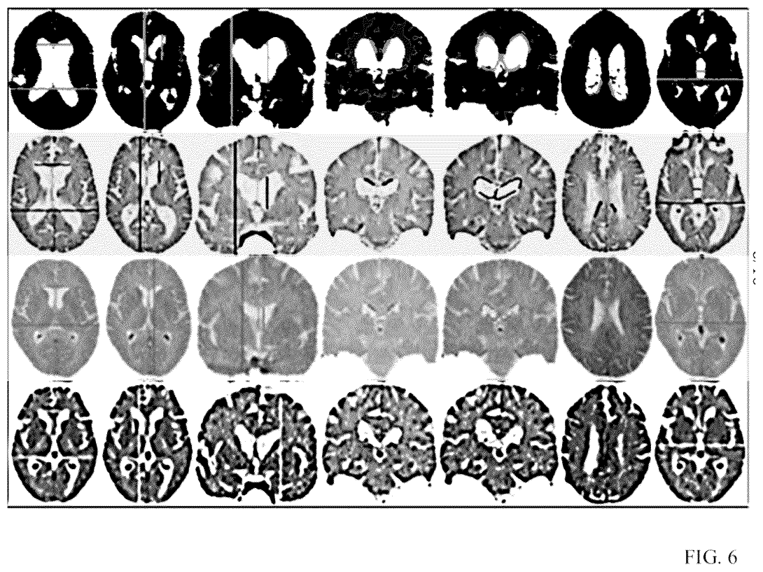

The measurement of 7 radiographic features on representative patient scans of NPH, AD, HC, and PT-cSDH is illustrated in FIG. 6. CSF2BVR is not shown as it is a ratio of the volume of CSF spaces to the total intracranial volume. On 115, 169, 64, and 80 NCCT scans of 89 NPH, 108 AD, 64 PT-cSDH, and 79 HC patients respectively, the measurements of EI-x, EI-y, EI-z, CA, MaxEccLV, p-SA, NMax-TVW, and CSF2BVR were successful in 95.5%, 99%, 99%, 92.7%, 95.7%, 90.3%, 98.8%, 100% of cases. Scans where all eight features were successfully computed are included for further analysis.

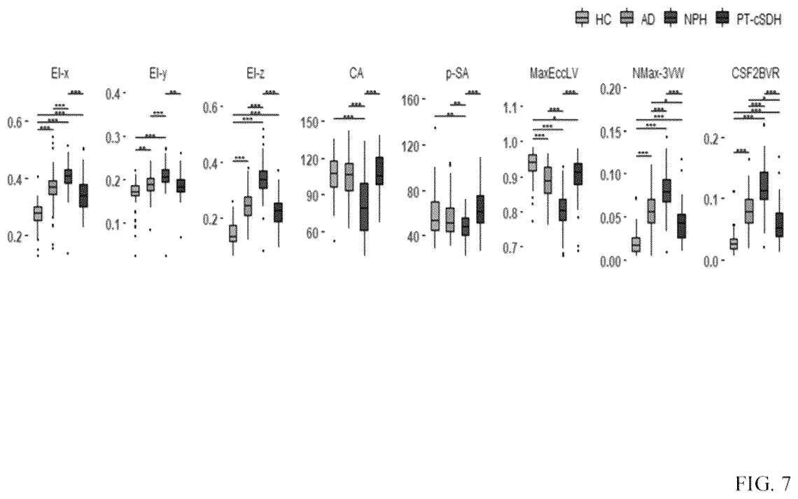

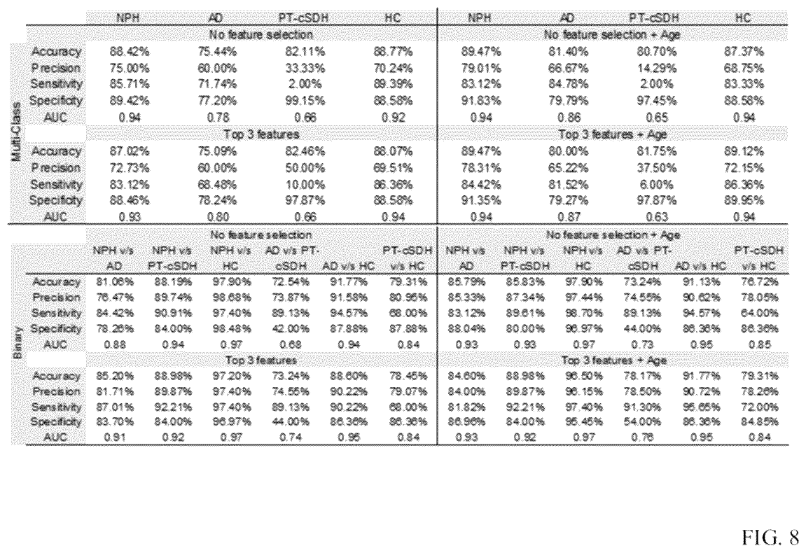

A boxplot visualizing the variation captured by these features and whether they were significantly different among the three groups is shown in FIG. 7. Significant differences were present in MaxEccLV (95% CIs: [0.12,0.14], [0.07,0.1], [0.08,0.12]), EI-x ([−0.14,−0.12], [−0.05,−0.03], [−0.08,−0.05]), EI-y ([−0.05,−0.03], [−0.03,−0.01], [−0.03,−0.01]), EI-z ([−0.21,−0.18], [−0.11,−0.08], [−0.14,−0.1]), CA ([19.09,31.41], [18.51,29.25], [20.05,32.89]), p-SA ([6.06, 16.68], [3.95,10.96], [9.94,21.19]), NMax-3VW ([−0.07,−0.05], [−0.03,−0.02], [−0.04,−0.03]), and CSF2BVR ([−0.1,−0.08], [−0.05,0.03], [−0.07,−0.05]) between NPH and the HC, AD, PT-cSDH cohorts respectively. The lateral ventricle size in terms of length, width, and height evidently increase in the order of HC, PT-cSDH, AD, and NPH cohorts as seen by the EI-y (0.17±0.03, 0.19±0.03, 0.19±0.02, 0.21±0.03), EI-x (0.27±0.04, 0.34±0.06, 0.37±0.05, 0.41±0.05), EI-z (0.14±0.04, 0.22±0.06, 0.25±0.05, 0.34±0.06) measurements. A similar trend is seen in NMax-3VW (0.02±0.01, 0.04±0.02, 0.06±0.02, 0.08±0.02), and CSF2BVR (0.03±0.02, 0.06±0.03, 0.08±0.03, 0.12±0.03). The trend observed in CA (107.930±15.850, 106.710±16.860, 105.340±15.780, 81.460±22.850) and p-SA (63.070±18.170, 58.870±19.710, 54.960±15.390, 47.50±11.360) is decreasing in the order of PT-cSDH, HC, AD, and NPH. The novel MaxEccLV (0.93±0.04, 0.9±0.06, 0.89±0.05, 0.8±0.05) decreases in the order of HC, PT-cSDH, AD, and NPH. Normalized features were used to fit logistic regression models to classify between NPH, AD, and HC. Repeated 5-fold cross validation was used for hyperparameter tuning such that the patients in train, test, and validation groups were mutually exclusive (FIG. 8).

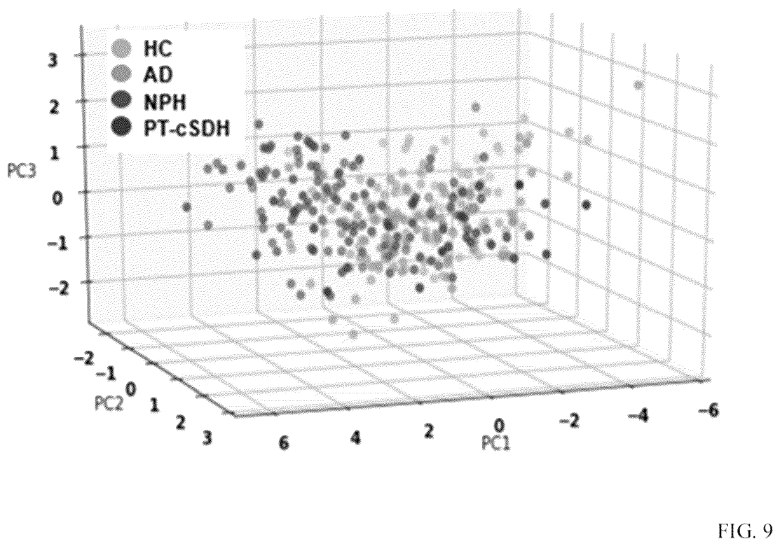

(FIG. 9) Random Forest Feature Importance was used to select the top 3 ventriculomegaly features to check for improvement in performance. The top 3 features according to random forest feature importance ranking were: EI-z, MaxEccLV, and CSF2BVR for the multi-class case and NPH v/s PT-cSDH; EI-z, MaxEccLV, and CA for NPH v/s AD; EI-x, EI-z, and CSF2BVR for NPH v/s HC, AD v/s HC, and PT-cSDH v/s HC; EI-x, NMax-3VW, and CSF2BVR for AD v/s PT-cSDH. Classification was repeated by including patient age (HC: 60 yrs.±12 mos., AD: 82 yrs.±7 mos., NPH: 76 yrs.±8 mos., PT-cSDH: 72 yrs.±13 mos.), in each case. Performance measures are reported by combining the class predictions on hold-out sets in the 5 repeats in Table 1 (FIG. 8). Assuming that HC can be compared to normal controls due to their negative CT, the NPH versus HC classification is better than the methodology developed so far [11,12] and is not impacted by age. We performed 3-dimensional Principal Component Analysis (PCA) on the data to show the separation between the groups in FIG. 9. It can be observed that there is near perfect separation between the NPH and HC cohorts.

In this work, we develop computational pipelines to extract eight different NCCT features suggestive of ventricular morphology. Image standardization is by a linear transformation, which does not introduce any brain size deformations which is undesirable when relative anatomical sizes need to be constant for comparative analysis. We introduce a novel feature called MaxEccLV which is among the top three features in distinguishing NPH from AD. Correlating this feature with the physical manifestation of NPH such as gait impairment might enable a better way to study the amount of WM impingement by the lateral walls of the ventricles. Classification results using a logistic regression model show that features of ventriculomegaly that are automatically derived from NCCT scans, can accurately differentiate NPH from AD, PT-cSDH, and HC. Adding age to the classification helped to improve the distinction between AD and the other groups as seen in the multi-class results as the patients were older but did not impact other classifications significantly. The nature of ventriculomegaly in PT-cSDH was unknown. The poor sensitivity in separating PT-cSDH in the multi-class classification case and the distribution of scans along the three PCs of ventriculomegaly features in PT-cSDH suggest that the ventriculomegaly in PT-cSDH is not sufficiently distinct. But it is evident from the binary classification metrics that there is good distinction between PT-cSDH and NPH in terms of ventriculomegaly, and the distinction gets weaker as it is compared to AD and HC. Our findings suggest that PT-cSDH may potentially involve a mostly atrophic component as opposed to a hydrocephalic component. The deep regression model for AC and PC landmark prediction enables localization of the significant landmarks on NCCT.

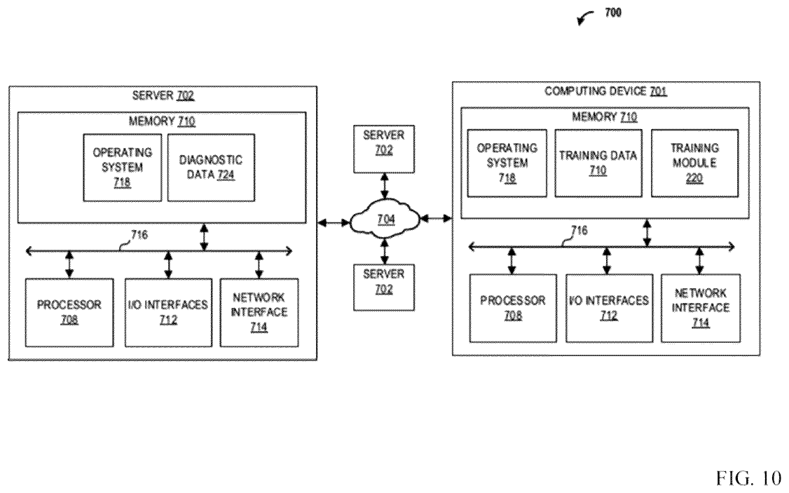

FIG. 10 is a block diagram depicting an environment 700 comprising nonlimiting examples of a computing device 701 and a server 702 connected through a network 704. In an aspect, some or all steps of any described method may be performed on a computing device as described herein. The computing device 701 can comprise one or multiple computers configured to store one or more of the neural networks, training data (e.g., labeled baseline data, labeled in-treatment data, and/or curated case agnostic data), and the like. The server 702 can comprise one or multiple computers configured to store data 724 (e.g., curated case agnostic data). Multiple servers 702 can communicate with the computing device 701 via the through the network 704.

The computing device 701 and the server 702 can be a digital computer that, in terms of hardware architecture, generally includes a processor 708, memory system 710, input/output (I/O) interfaces 712, and network interfaces 714. These components (708, 710, 712, and 714) are communicatively coupled via a local interface 716. The local interface 716 can be, for example, but not limited to, one or more buses or other wired or wireless connections, as is known in the art. The local interface 716 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 708 can be a hardware device for executing software, particularly that stored in memory system 710. The processor 708 can be any custom made or commercially available processor, a central processing unit (CPU), an auxiliary processor among several processors associated with the computing device 701 and the server 702, a semiconductor-based microprocessor (in the form of a microchip or chip set), or generally any device for executing software instructions. When the computing device 701 and/or the server 702 is in operation, the processor 708 can be configured to execute software stored within the memory system 710, to communicate data to and from the memory system 710, and to generally control operations of the computing device 701 and the server 702 pursuant to the software.

The I/O interfaces 712 can be used to receive user input from, and/or for providing system output to, one or more devices or components. User input can be provided via, for example, a keyboard and/or a mouse. System output can be provided via a display device and a printer (not shown). I/O interfaces 712 can include, for example, a serial port, a parallel port, a Small Computer System Interface (SCSI), an infrared (IR) interface, a radio frequency (RF) interface, and/or a universal serial bus (USB) interface.

The network interface 714 can be used to transmit and receive from the computing device 701 and/or the server 702 on the network 704. The network interface 714 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 714 may include address, control, and/or data connections to enable appropriate communications on the network 704.

The memory system 710 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.) that are non-transitory. Moreover, the memory system 710 may incorporate electronic, magnetic, optical, and/or other types of storage media. Note that the memory system 710 can have a distributed architecture, where various components are situated remote from one another, but can be accessed by the processor 708.

The software in memory system 710 may include one or more software programs, each of which comprises an ordered listing of executable instructions for implementing logical functions. In the example of FIG. 7, the software in the memory system 710 of the computing device 701 can comprise the training module 220 (or subcomponents thereof), the training data 210B, and a suitable operating system (O/S) 718. In the example of FIG. 10, the software in the memory system 710 of the server 702 can comprise, the data 724, and a suitable operating system (O/S) 718. The operating system 718 essentially controls the execution of other computer programs and provides scheduling, input-output control, file and data management, memory management, and communication control and related services. One or more of the components may be used in an endoscope.

For purposes of illustration, application programs and other executable program components such as the operating system 718 are illustrated herein as discrete blocks, although it is recognized that such programs and components can reside at various times in different storage components of the computing device 701 and/or the server 702. An implementation of the neural network and training process can be stored on or transmitted across some form of computer-readable media. Any of the disclosed methods can be performed by computer-readable instructions embodied on computer-readable media. Computer-readable media can be any available media that can be accessed by a computer. By way of example and not meant to be limiting, computer readable media can comprise “computer storage media” and “communications media.” “Computer storage media” can comprise non-volatile, removable and non-removable media implemented in any methods or technology for storage of information such as computer readable instructions, data structures, program modules, or other data. Example computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer. The computing device 701 may be used train neural networks (e.g., convolutional neural networks) described herein. For example, training data may be curated and used to train and test the neural networks. The trained neural networks may be used to perform one or more steps, processes, or methods, described herein.

### Example 1

In one example, a computational pipeline was developed to extract objective features of ventriculomegaly from Non-Contrast CT (NCCT) for the accurate classification of idiopathic Normal Pressure Hydrocephalus (NPH) from headache controls (HC), Alzheimer's Dementia (AD), and post-traumatic encephalomalacia (PTE).

In one example, patients with possible NPH (NPH, n=79) and a subset with definite NPH (DefNPH, n=29) were retrospectively identified on the Veterans Affairs Informatics and Computing Infrastructure, along with AD (n=62), PTE (n=53), and HC (n=59) cohorts. Image processing pipelines were developed to extract a novel feature capturing lateral ventricle eccentricity (MaxEccLV), a proxy-Splenial Angle (p-SA), the Evans indices (EI-x,y,z), Callosal angle (CA), third ventricle width (NMax-3VW), and cerebrospinal fluid (CSF) to brain volume ratio (CSF2BVR) from their NCCT scans. T-tests were used to examine group differences in the features, and logistic regression models for classification. Additionally, the NPH v/s HC classifier was validated on external data.

When NPH and DefNPH were compared to HC/AD/PTE, significant differences were seen in all features except the p-SA which only significantly differed in PTE. The test-set AUC, sensitivity, and specificity were 0.98, 100%, and 98.3% for NPH v/s HC; 0.94, 87.3%, and 85.5% for NPH v/s AD; 0.96, 92.4% and 90.6% for NPH v/s PTE; and 0.96, 94%, and 88% for NPH v/s rest using logistic regression under 5-fold cross validation. Consistently high performance was noted for DefNPH. The NPH v/s HC classifier provided an AUC of 0.84, sensitivity of 76.9%, and specificity of 90% when assessed on external data.

Including the novel MaxEccLV, the framework computes useful features of ventriculomegaly, which had not been algorithmically assessed on NCCT so far. It successfully classified possible and definite NPH from HC/AD/PTE. Following validation on larger representative cohorts, this objective and accessible tool may aid in screening for NPH and its differentiation from symptomatic mimics like AD/PTE.

Improved screening of idiopathic Normal Pressure Hydrocephalus (NPH) is crucial as it may be reversed with shunt-surgery, with earlier intervention leading to better outcomes. Despite contrary evidence, reported incidence rates of NPH are very low, ranging from 1.19 to 1.8 per 100,000 persons, suggesting its under-recognition. Distinguishing NPH from its mimics is also important for better therapeutic intervention. Less than 20% of NPH patients received accurate diagnosis with most cases attributed to Alzheimer's Dementia (AD) or other neurological diseases due to overlapping symptoms like gait impairment and dementia. Structurally, ventriculomegaly manifests due to hydrocephalus in NPH and atrophy in AD. Chronic effects of neurotrauma such as subdural hemorrhages (SDH) are also reported to cause gait impairment and dementia. Hydrocephalus is a known secondary complication of head trauma and atrophy occurs in chronic SDH (cSDH) at rates higher than dementia. This overlap of structural and symptomatic effects indicates that an algorithmic evaluation of NPH may be of substantial assistance to radiologists and clinicians.

Even though MRI is preferred due to higher resolution, any patients experience falls as a first indicator of a neurological condition, and Non-Contrast Computed Tomography (NCCT) scans are routinely acquired after falls in emergency settings. NCCT scans are more affordable, quicker, and have fewer contraindications as compared to MRI, which makes them an excellent source to screen for potential NPH. Currently, the Evans Index is the only standard diagnostic measure of NPH that has been automatically computed from NCCT. The SILVER index was proposed to quantify the Disproportionately Enlarged Subarachnoid Space Hydrocephalus (DESH) which is a prominent feature of NPH. And the iNPH Radscale incorporates a visual grade of DESH, but these methods involve manual evaluation which is subject to observer variability. While computational formulations of such measures are plausible, they lack the inherently objective definition of linear ventricular indices and angles. Recently, automated segmentation of NCCT scans integrated with inferred connectome data and a convolutional neural network (CNN) was used to classify NPH from controls but computational methods to measure interpretable features are still lacking. Additionally, NPH has been distinguished from AD using manually measured CA and EI-x measures and white matter (WM) changes on MRI, but there have been no (automatic) NCCT-based methods. The distinction of ventricular morphology between NPH and post-traumatic encephalomalacia (PTE) secondary to cSDH also remains unknown.

While clinical diagnosis of NPH should rely on examining multi-modal data, a non-invasive screening methodology based on computational assessment of NCCT scans may aid radiologists and clinicians in reducing misdiagnoses and thus treatment. Such a method could also detect shunt-responsive NPH and distinguish it from its mimics. Here, a computational pipeline is developed to extract eight features of ventriculomegaly—a novel feature which we define to be the Maximum Eccentricity of the lateral ventricles (LVs) (MaxEccLV), a proxy-SA (p-SA), the Evans Indexx (EI-x), Evans Indexy (EI-y), Evans Index, (EI-z), Callosal Angle (CA), Normalized Maximum Third Ventricle Width (NMax-3VW), and CSF to brain volume ratio (CSF2BVR), using image processing. This shows the utility of their computational assessment in detecting NPH and distinguishing it from its mimics by applying them to classify possible and definite NPH from AD, PTE, and HC. Feature specific differences are also examined among the disease groups and validate the NPH v/s HC classifier using external data.

### Methods:

### Cohort Selection

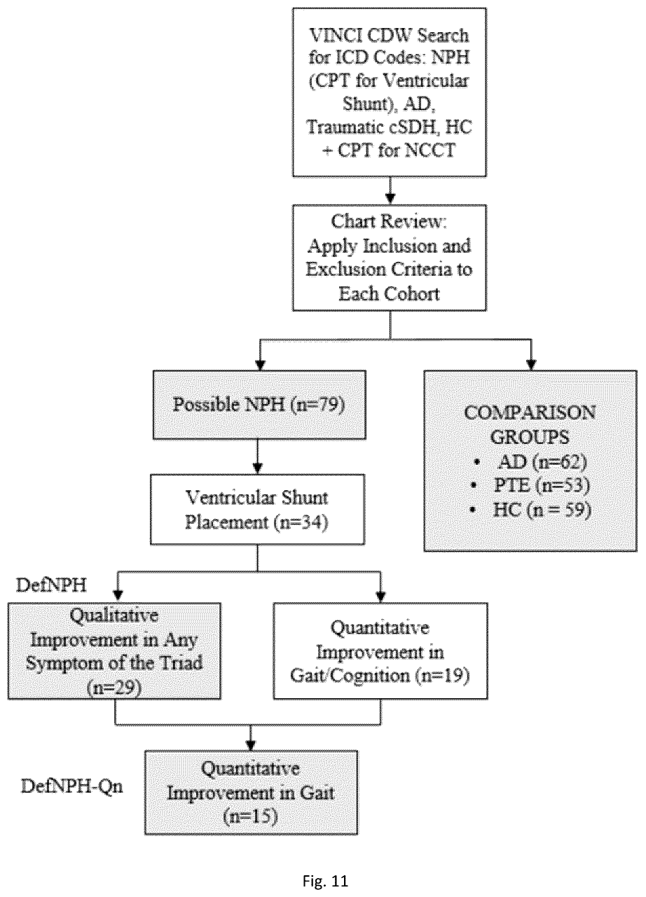

IRB approval was obtained from the Minneapolis Veterans affairs (VA) with an informed consent waiver (#4593-B). We queried the VA Networking and Communication Infrastructure (VINCI) system for patients who obtained an NCCT scan in any of the Midwest care locations from 2000-2022. NPH, AD, traumatic-cSDH, and headache (HC) diagnoses were initially inferred by International Classification of Disease codes. Patient charts including but not limited to neurology, neurosurgery, neuropsychology, geriatric/primary care, physical/occupational therapy, and discharge summary reports were reviewed to validate diagnoses. FIG. 11 summarizes the cohort selection process.

The 3^{rd} edition of the Japanese Guidelines was applied to qualify the possible NPH cohort (n=79). Among them, we found records of shunt-surgery for 34 patients. The guidelines characterize definite NPH as a positive shunt (and/or reprogramming) response, and comment on various tests that can be applied to characterize it. Different approaches are taken in existing studies, as there is no single recommended measure of improvement. In this study, patients with improvements noted in any symptom of the NPH triad post-surgery, by their neurologist/neuropsychiatrist/neurosurgeon, occupational/physical therapist, geriatric care/primary-care specialist, or self/family were categorized as definite NPH. 29 patients (DefNPH) were found characterized by such qualitative improvement, and 27 had improved gait. Due to the retrospective nature of this study, records of quantitative gait and cognitive tests were not available for all shunted patients, and not uniform across patients who had them. Therefore, a subset with the same pre- and post-op gait/cognitive test were assessed for quantitative improvement. In the DefNPH group, 19 patients had quantitative improvement in gait/cognition, and 15 of them had quantitative gait improvement. In the remaining 4, improvement was minimal in cognition and absent in incontinence. Subsequently, we focused on analyzing the possible NPH (NPH, n=79) group, its subset of shunted patients who had qualitative (DefNPH, n=29) and quantitative (DefNPH-Qn, n=15) improvements.

Diagnosis of AD, traumatic-cSDH (PTE), and headache (HC) were confirmed on patient charts. The PTE scans were visually examined to ensure absence of acute hemorrhage that may have required surgical intervention, and scans of NPH patients were confirmed to be pre-op. The exclusion criteria that were applied to all cohorts was history/presence of meningitis, encephalitis, tumor, Parkinson's Disease, Aqueductal stenosis, Progressive Supranuclear Palsy, Dementia with Lewy Bodies, Frontotemporal dementia, Vascular Dementia, intracranial hemorrhage except SDH, significant stroke, or any other neurological disorder that could clearly explain symptoms. The NPH (n=79), AD (n=62), PTE (n=53), and HC (n=59) cohorts were confirmed to be mutually exclusive.

### Power Analysis

Power analysis using G*Power (3.1.9.7) indicated a total sample size of n=109 to reject the null hypothesis that the squared multiple correlation between a response variable (NPH/non-NPH) and a set of eight predictors is zero, with a medium effect size and power of 80%. With a set of three predictors, it indicated a total sample size of n=77. According to this, our NPH, AD, PTE, and HC cohorts were of appropriate sizes for two-class comparisons.

### NCCT Scan Parameters

All scans were acquired on Philips, Siemens, or Toshiba scanners. Most scans were acquired with a peak tube voltage of 120 kVp, with a few acquired at 100 kVp, 130 kVp, and 140 kVp. The gantry-tilts varied from −22 to +15 degrees, slice thickness from 0.625 mm to 5 mm, and X-ray tube current from 54 to 500 mAs. The median number of slices was 32 and the median pixel spacing was 0.47 mm×0.47 mm.

### Image Preprocessing and Ventricle Segmentation

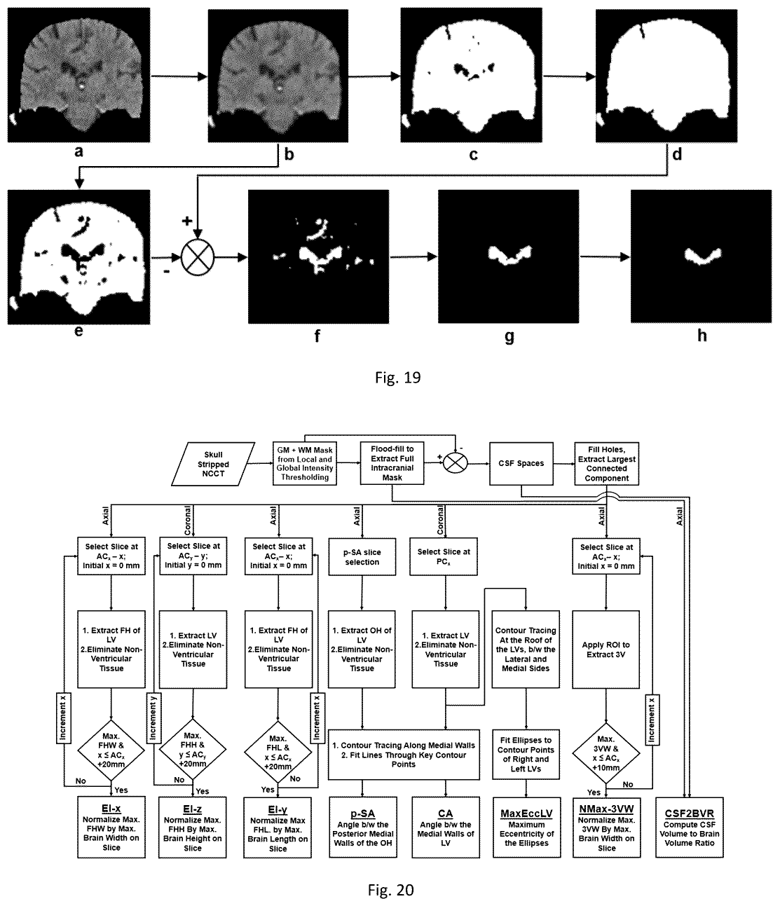

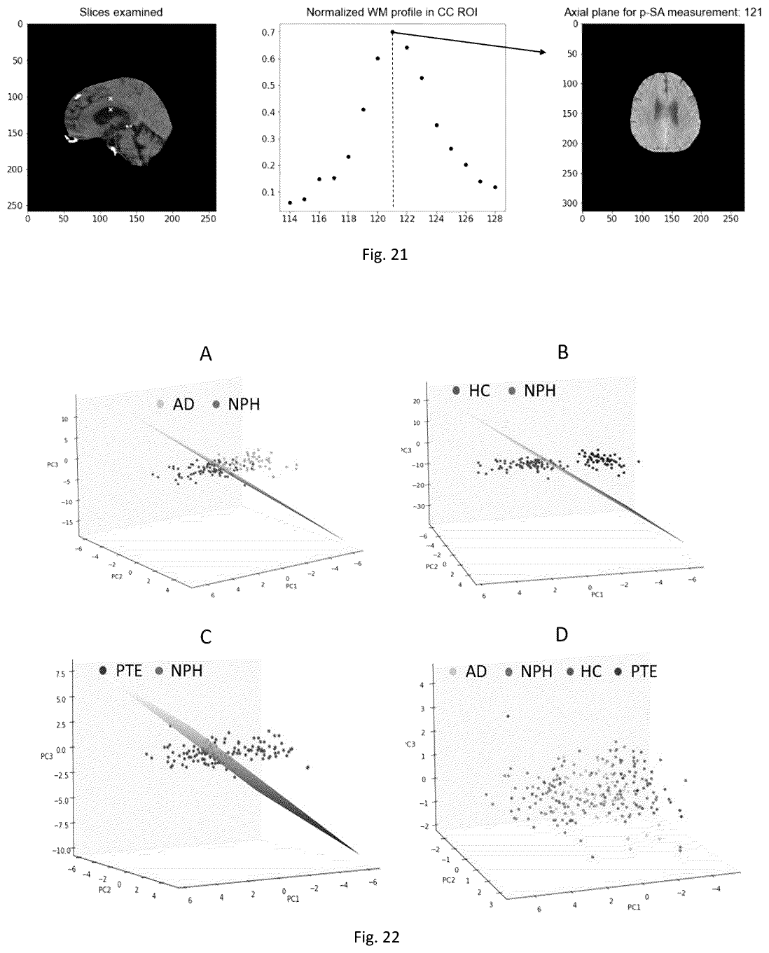

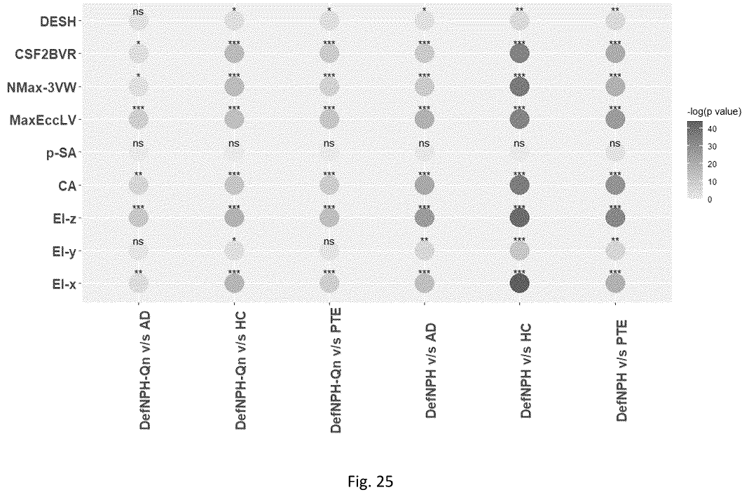

3D Slicer, OpenCV, and Simple Insights Toolkit (SITK) with Python 3.7.3 were used for all image processing methods. The preprocessing steps were gantry-tilt correction, isotropic resampling, bed removal, and skull stripping. The anterior commissure (AC), posterior commissure (PC), and midline were manually annotated on all scans. A linear standardizing transformation was then applied to resample the images such that the line connecting the AC and PC was horizontal and the midline was vertical. Ventricle segmentation is a prerequisite for the computation of our radiographic features. We took a simpler density-based segmentation approach to mitigate unnecessarily high computational load and post-processing required by advanced methods. This is illustrated in Supplemental FIG. 19, and detailed in the Supplementary Methods and Results (FIGS. 19-25). Apart from the manual annotation of the AC, PC, and midline, this framework is automatic.

### Measurement of Ventriculomegaly Features

Supplemental FIG. 20 illustrates the computational pipeline to extract the eight ventriculomegaly features, following the general segmentation approach mentioned above. Feature-specific details are below.

MaxEccLV and p-SA

Based on the observation that the LVs of patients who suffered from NPH had a “rounder” appearance on the standard coronal plane at the level of the PC, we hypothesized that the maximum eccentricity of ellipses that approximate the left and right LVs (LLV and RLV), MaxEccLV, would be smaller in NPH patients. Our rationale for this novel feature was that it quantifies the 2D enlargement of the LVs, with lower values indicating more hydrocephalus.

The Splenial Angle (SA) is measured using diffusion tensor MRI, calculated as the angle subtended by the forceps major at the midline, and shown to differentiate NPH from AD and normal controls. As WM is poorly imaged on NCCT, we defined a proxy measure called the proxy-SA (p-SA) as the angle between the medial walls of the occipital horns (OHs) of the LVs as imaged on the first axial plane with the body of the CC completely visible while scrolling in the inferior-superior direction. Based on the observation that the forceps-major follow the medial walls of the OHs of the LV and that the ventricles are easier to segment on NCCT than WM, we expected this measure to be a reasonable proxy to the SA.

The computational details for these features are in the Supplementary Methods and Results.

### EI-x, EI-y, EI-z, CA, NMax-3VW, and CSF2BVR

The computation of these features is based on previously defined measures that have been shown to capture NPH. EI-x is the maximum frontal horn width (MFHW) of the LVs normalized by the brain width (BW), and CA is the angle between the LVs as measured on the standard coronal plane at the level of the PC. EI-y is computed as the maximum frontal horn length (MFHL) normalized by the brain length (BL), and EI-z as the maximum frontal horn height (MFHH) normalized by the brain height (BH). The ratio between CSF volume to brain volume is likely to be higher in NPH than in atrophy; we denote it by CSF2BVR. The third ventricle (3V) is also reported to be enlarged in NPH. We extracted the maximum 3V width (M3VW) normalized by BW and denoted it by NMax-3VW. The standard range of axial slices required to measure the EI-x, EI-y, and NMax-3VW were determined by the location of the AC, and that of coronal slices required to measure the EI-z was determined by the PC.

### Statistical Analysis

### Group Differences and Feature Importance

Two-sided, two-sample t-tests for means were used to evaluate the group differences among all the features by considering each pair of disease cohorts at a confidence level of 95%. We applied Bonferroni correction to reduce false discovery rate. Significant differences as indicated adjusted p-values and confidence intervals are reported for each test per feature.

### Classification

### Classification of Internal Veteran Data

The NPH, AD, PTE, and HC were classified in multi-class (one-versus-all) and (six) binary classification scenarios using logistic regression (LR) models. For each case, we split the data into training (80%) and test (20%) sets and normalized both with the training set's mean and standard deviation. LR models trained on the training set with default hyper-parameters were used to examine permutation feature importance (PFI). Each feature was permuted randomly 100 times to record the drop in test-set accuracy, and the top 3 impactful features were noted. Using another 80%-20% (training-cv, validation-cv) split on the training set alone, hyper-parameters (regularization type and strength) of the LR models were optimized using the validation-cv set's accuracy, under 5-fold cross validation (CV). The classification metrics including accuracy, sensitivity, precision, specificity, and area under the curve of receiver operating characteristics (AUC) were calculated by aggregating the previously held out test set predictions for each fold. This was repeated with the top 3 features, and with and without age. We tested linear and nonlinear kernels using support vector machines (SVMs) for potential improvement in classification.

The DefNPH and DefNPH-Qn cohorts were also classified from the rest (AD/PTE/HC) in multi-class and binary settings in the manner described above.

Validation of the NPH v/s HC Classifier with External Cohorts

To evaluate the generalization that our NPH v/s HC classifier provides, the model trained on our data was used to classify between possible NPH (External-NPH) and normal controls (External-Control) on a dataset provided to us by Zhang et al.'s group at the UCSB. These scans were processed through our framework to obtain the eight features of ventriculomegaly. The NPH v/s HC classifier trained on our data was used to classify between these cohorts, and results were reported under 5-fold CV.

### Examining the Effect of Age-Matching

We examined the effects of age-matching on our classification using the MatchIt package (4.5.2) in R, by selecting age-matched subsets of each cohort and repeating the classification. We used Python 3.7.3 and RStudio 4.1.0 for analysis.

### Results:

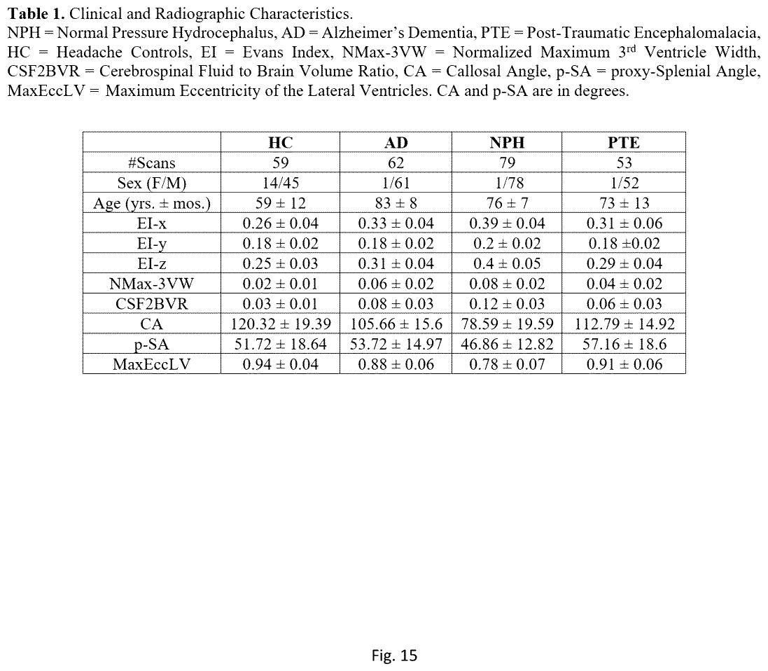

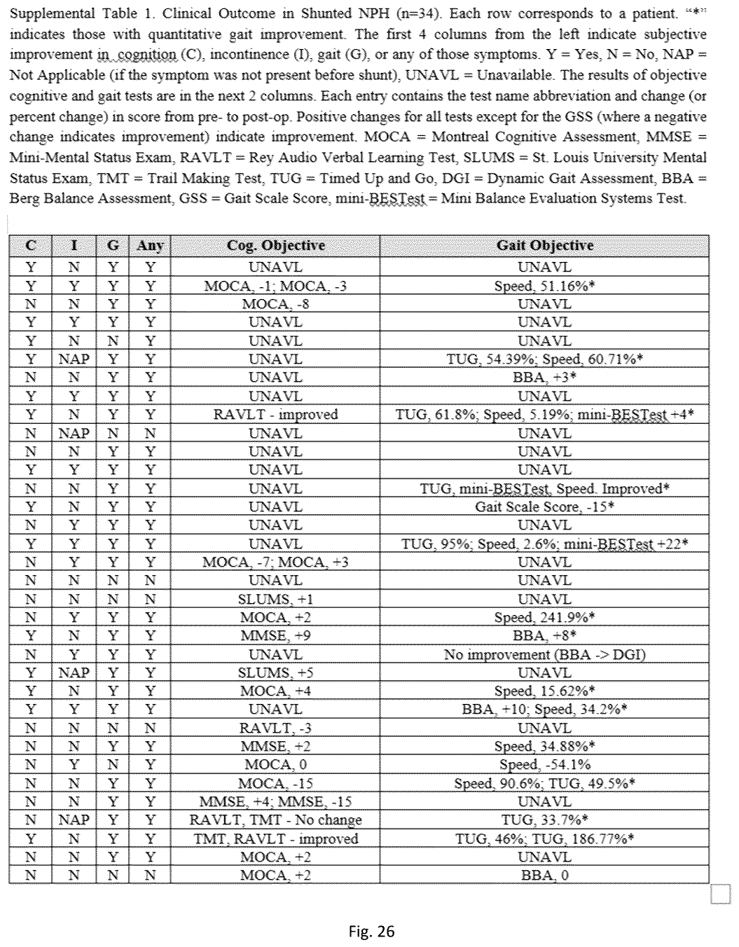

FIG. 12 shows the key measures that the pipeline identified to calculate all the measures except CSF2BVR. Table 1 (FIG. 15) contains the clinical and radiographic characteristics of the veteran cohort. The AD cohort was the oldest, followed by NPH, PTE, and HC. More than 90% of the patients were male. The EI-x, EI-y, EI-z, NMax-3VW, and CSF2BVR decreased in the order of NPH, AD, PTE, and HC. The CA and MaxEccLV show an opposite trend increasing in the order of NPH, AD, PTE, and HC. The p-SA deviated from this trend, decreasing in the order of PTE, AD, HC, and NPH. The post-shunt outcomes of the 34 shunted NPH patients are provided in Supplemental Table 1 (FIG. 26).

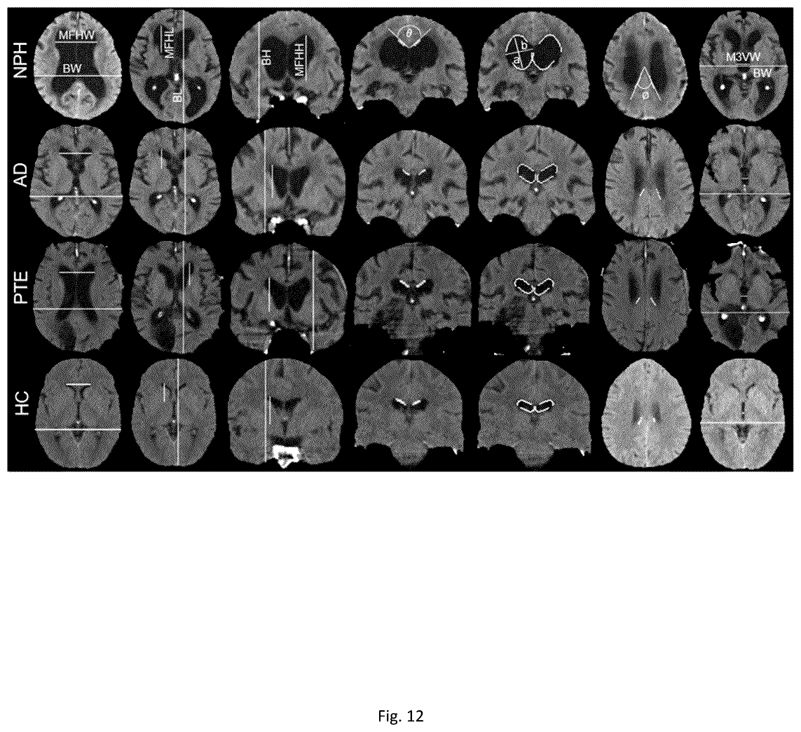

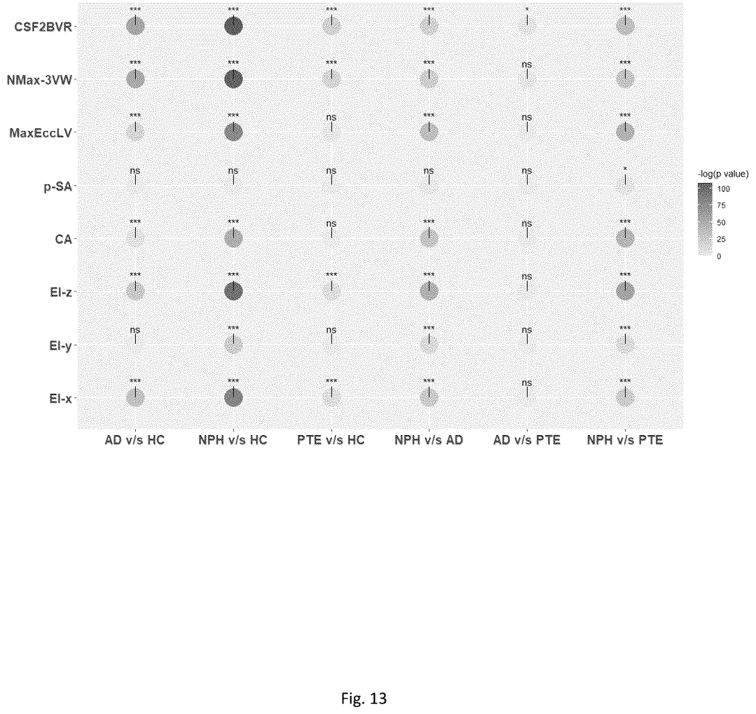

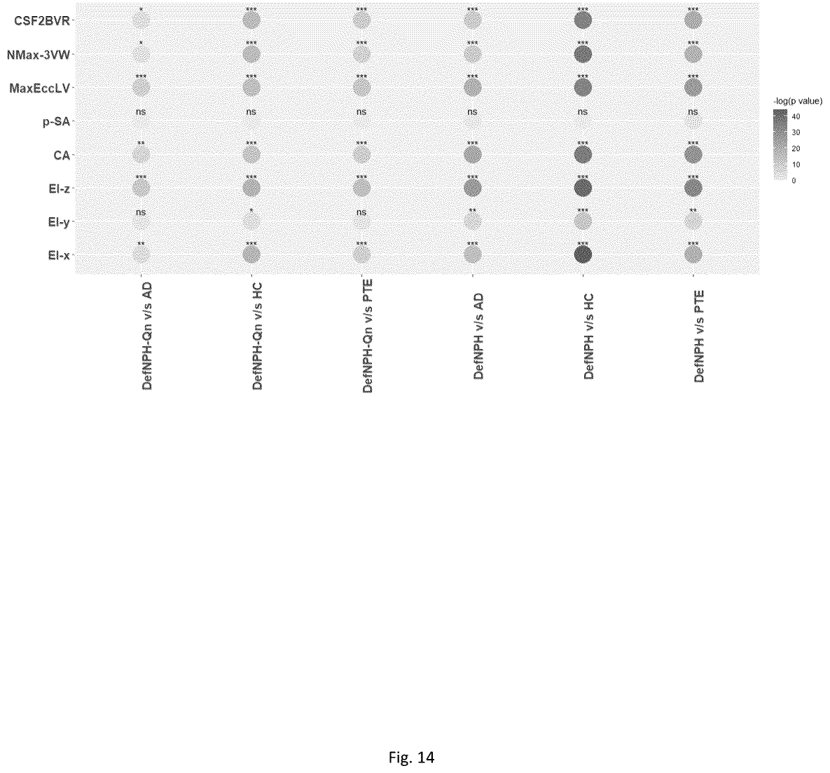

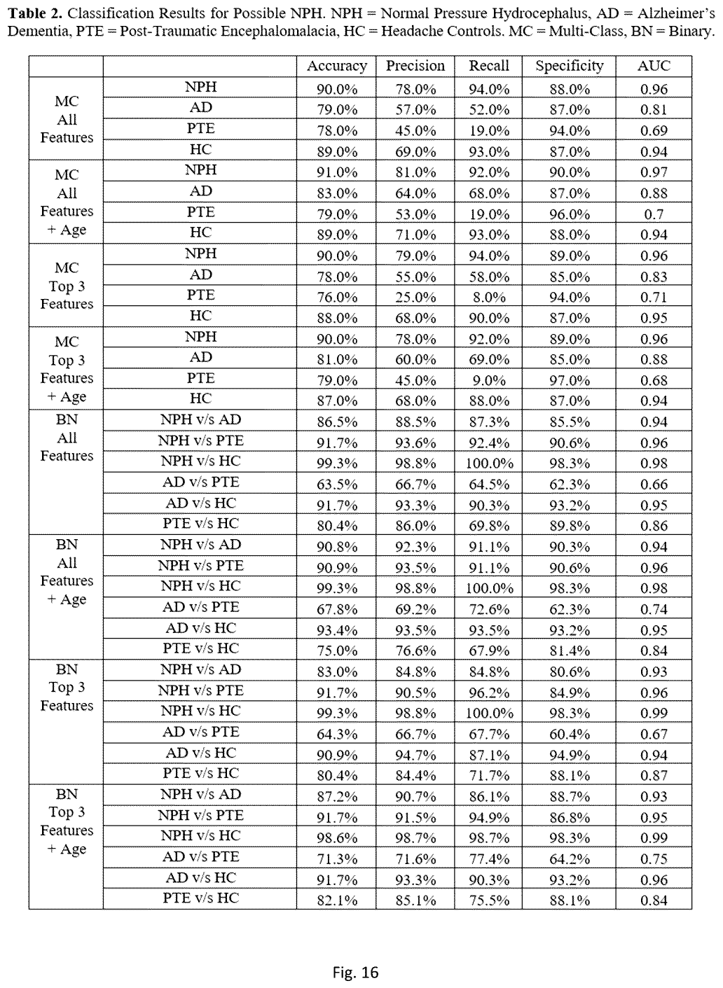

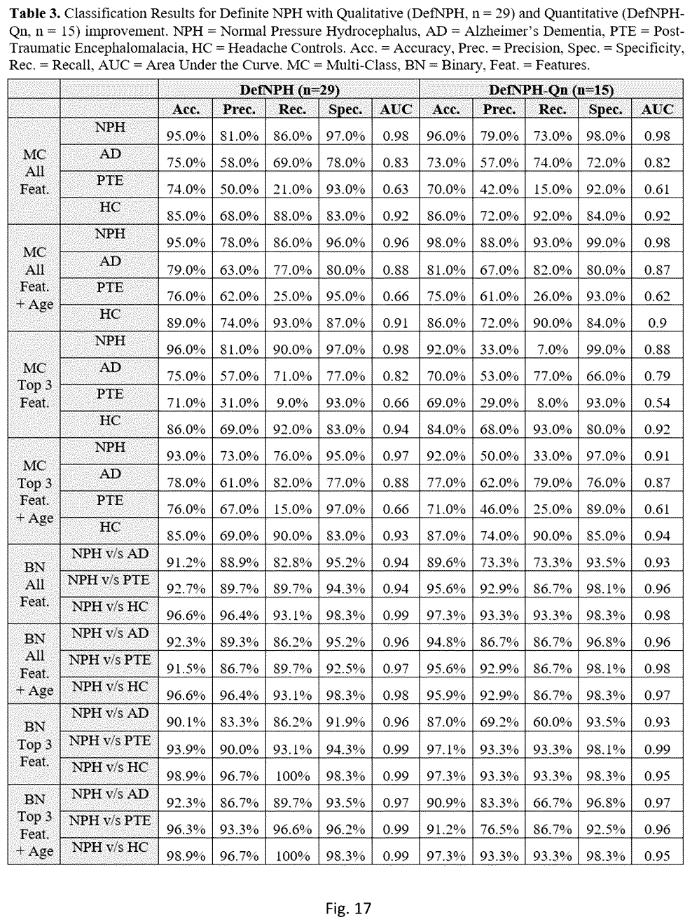

Table 2 (FIG. 16) contains the classification results of NPH from the rest for both multi-class and binary scenarios using LR models. Table 3 (FIG. 17) contains the same for the DefNPH/DefNPH-Qn cohorts. SVMs with linear or nonlinear kernels did not perform better than LR. A scatterplot of all patients visualized in 3D-space reduced from the 8D-space of the ventriculomegaly features is in Supplemental FIG. 22. FIGS. 13-14 indicates significant differences between NPH and AD/PTE/HC for each feature, measured as the negative logarithm of the adjusted p-value obtained from t-tests for means. The same for DefNPH/DefNPH-Qn are shown in FIG. 14. The key results are below.

### Multiclass Classification:

In the multi-class scenario, NPH was distinguished from the rest with an AUC of 0.96, sensitivity of 94% and specificity of 88% by using all the eight features, and there was minimal impact with feature selection and adding age. AD's distinction from the rest was poor but using only the top 3 features selected by PFI (EI-z, CSF2BVR, CA) improved the sensitivity by 6 points, and adding age to them notably increased the sensitivity by 16 points, resulting in an AUC of 0.88, sensitivity of 68%, and specificity of 87%. PTE was poorly classified with an AUC of 0.69, sensitivity of 19%, and specificity of 94%, and the sensitivity did not improve with age or feature selection. DefNPH was also very well distinguished from the rest with an AUC of 0.98, sensitivity of 86% and specificity of 97%. The AUC and specificity were unaffected when DefNPH-Qn was classified from the rest, with a reduced sensitivity of 73%.

### Binary Classification:

### NPH v/s HC

Between NPH and HC, the t-test indicated all features as significantly different except the p-SA, ranked in the order listed: CSF2BVR (95% CI: [0.08,0.1]), NMax-3VW ([0.06,0.07]), EI-z ([0.13,0.16]), EI-x ([0.12,0.15]), MaxEccLV ([−0.18,−0.14]), CA ([−48.36,−35.1]), and EI-y ([0.02,0.03]). Significant differences were in these features when DefNPH and DefNPH-Qn were compared to HC. NPH was classified from HC with an AUC of 0.98, sensitivity of 100%, and specificity of 98.3%. DefNPH was consistently well classified from HC using the top 3 features selected by PFI (NMax-3VW, EI-z, EI-z). DefNPH-Qn v/s HC had an AUC of 0.98, sensitivity of 93.3%, and specificity of 98.3%. Feature selection did not impact performance.

### External Validation of the NPH v/s HC Classifier

The classifier trained on our NPH and HC veteran dataset classified the External-NPH (n=13, 79 yrs.±7 mos.) and External-Control (n=30, 73 yrs.±9 mos) cohorts with an AUC of 0.84, a sensitivity of 76.9%, and a specificity of 90%.

### NPH v/s AD

All features were significantly different between NPH and AD except for the p-SA, ranked in the order listed: EI-z ([0.08,0.11]), MaxEccLV ([−0.12,−0.08]), CA ([−32.93,−21.21]), EI-x ([0.05,0.07]), NMax-3VW ([0.02,0.03]), CSF2BVR ([0.02,0.04]), and EI-y ([0.01,0.03]). Significant differences between DefNPH/DefNPH-Qn and AD were in those features except in the EI-y between DefNPH-Qn and AD. NPH was classified from AD with an AUC of 0.94, sensitivity of 87.3%, and specificity of 85.5%. Including age increased the sensitivity by 4 points and specificity by 5 points. Feature selection did not have a marked effect. DefNPH was classified from AD with a sensitivity of 86.2%, specificity of 91.9%, and AUC of 0.96, using the top 3 features selected by PFI (EI-z, MaxEccLV, and CSF2BVR). DefNPH-Qn was classified from AD with a sensitivity of 73.3%, specificity of 93.5%, and AUC of 0.93. As in the NPH v/s AD case, adding age increased the sensitivity and feature selection did not have a marked effect.

### NPH v/s PTE

NPH and PTE showed a significant difference in all features, ranked in the order listed: EI-z ([0.9,0.13]), MaxEccLV ([−0.15,−0.11]), CA ([−40.15,−28.24]), CSF2BVR ([0.04,0.06]), NMax-3VW ([0.03,0.05]), EI-x ([0.06,0.1]), EI-y ([0.01,0.03]), and p-SA ([−16.13,−4.47]). Significant differences between DefNPH/DefNPH-Qn and PTE were in those features except in the p-SA between DefNPH and PTE, and the p-SA and EI-y between DefNPH-Qn and PTE. This group was distinguished with a high AUC of 0.96, sensitivity of 92.4%, and specificity of 90.6%. Feature selection and adding age did not have a marked effect. DefNPH was classified from PTE with a sensitivity of 93.1%, specificity of 94.3%, and AUC of 0.99, using the top 3 features selected by PFI (EI-z, CA, and MaxEccLV). DefNPH-Qn was similarly well distinguished from PTE using the top 3 features selected by PFI (same as those for DefNPH).

The classification results for comparisons among the AD/PTE/HC cohorts are in the Supplementary Methods and Results.

Classification after Age-Matching

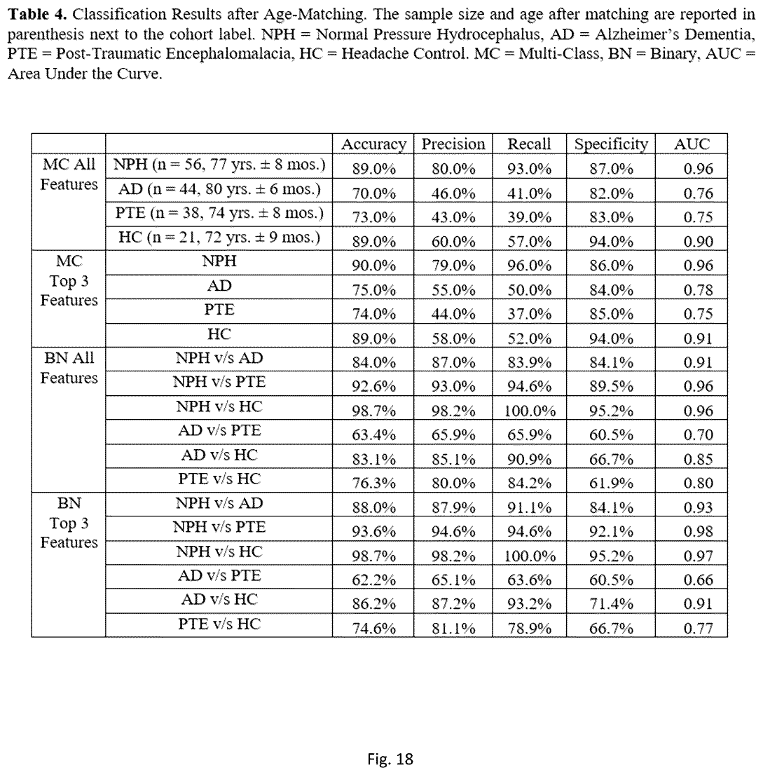

Table 4 (FIG. 18) contains the classification results post age-matching. There was improvement in the recall by 4 points when NPH is classified from AD using the top 3 features selected by PFI (CSF2BVR, EI-z, and MaxEccLV). The NPH v/s PTE, HC classifications remained relatively unaffected. The distinction between NPH v/s AD/PTE/HC in a multi-class setting is also largely unaffected.

### Discussion

Here, we developed a computational pipeline to extract eight features of ventriculomegaly including the novel MaxEccLV, a CT based proxy for the SA, and five prominent features (the CA, EI-y, EI-z, NMax-3VW, and CSF2BVR) which have not been algorithmically assessed on NCCT before. It is automated except for the manual annotation of the standard landmarks of AC, PC, and midline, which makes it easily applicable for use in clinical settings. The EI-x has been automatically extracted before, using nonlinear registration which renders the brain width almost constant and impacts measurement. Linear standardization in our methodology keeps relative anatomical sizes constant for accurate comparative analysis. Automating linear measurements of the maximum third ventricle width and frontal horn (of the lateral ventricles) width on NCCT has also been attempted with ResNet models, but it does not capture any variation in the z-direction and lacks the deterministic nature of our pipeline.

Interpretable trends observed in these features correlate with known structural effects of these diseases. MaxEccLV suggests that “rounder” ventricles are more suggestive of hydrocephalic pathology. As it measures overall ventricular expansion, we hypothesize that it may be correlated with gait impairment, arising from the force exerted by the LVs onto surrounding WM. Further investigation is needed to establish its clinical significance. Even though the p-SA only captured significant differences between NPH and PTE, the trend observed in it with smaller angles in NPH as opposed to the rest is consistent with the SA which is originally measured between the forceps major WM tracts. The trend in CA, EI-x, EI-y, EI-z, NMax-3VW, and CS2BVR are also consistent with previous findings.

The excellent distinction between the NPH and HC cohorts on NCCT is at par with evaluation using MRI. This was also reflected in patients with definite NPH, and those with quantitative gait improvement. Age-matching the cohorts resulted in consistently high performance. We also observed that our model can distinguish between an external NPH and normal cohort with reasonable sensitivity and specificity, underscoring its generalizability. While evaluation on a larger definite NPH cohort with standardized quantitative outcome is required to rigorously assess our framework against manual NCCT assessment standards and more recent methods using deep learning that predicted definite NPH from asymptomatic individuals, its applicability as a screening tool in detecting possible NPH using objective and interpretable features that can aid radiologists in decision making is well established. In addition, we show that definite NPH was also well distinguished from the HC cohort, indicating the potential of this framework's clinical applicability in predicting shunt-responsive NPH.

We reported a high distinction between NPH and AD, with a high AUC, sensitivity, and specificity. To the best of our knowledge, there are no previous automated efforts to use NCCT scans for this purpose. Age-matching these cohorts led to increased sensitivity which may be reflective of older AD patients with higher atrophy resulting in higher ventriculomegaly getting eliminated. NPH is also well separated from the AD/PTE/HC cohorts in a one-vs-all case. The poor sensitivity in separating PTE in the multi-class classification may suggest that ventriculomegaly in PTE is not sufficiently distinct. The fact that NPH was well classified from PTE with high sensitivity and specificity, but the AD versus PTE classifier showed low specificity suggests that ventriculomegaly in PTE may be more atrophic than hydrocephalic (Supplemental FIG. 22).

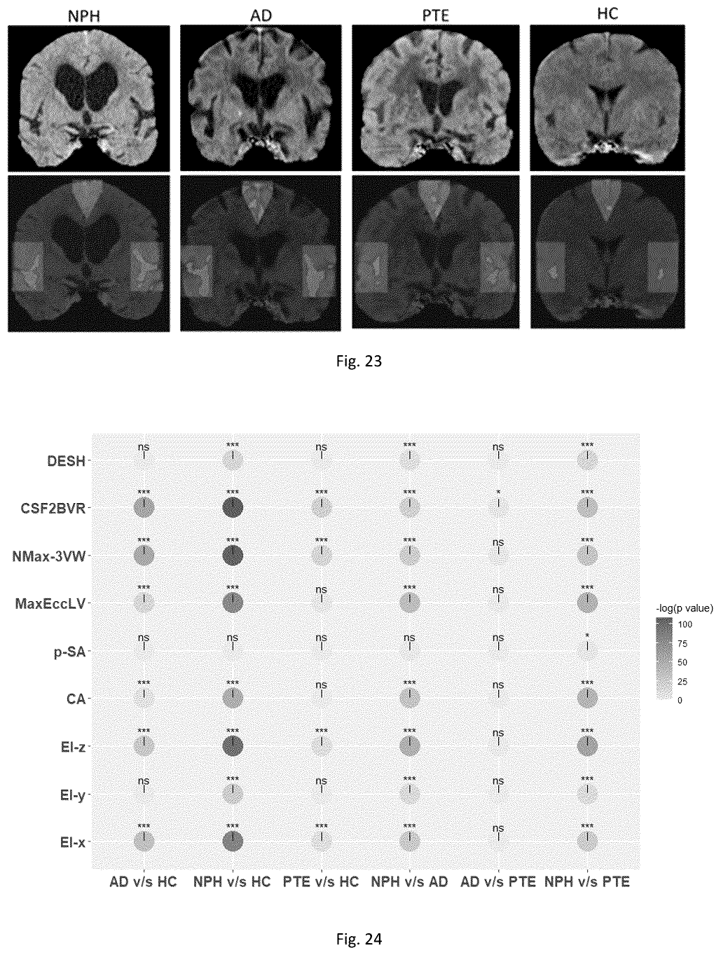

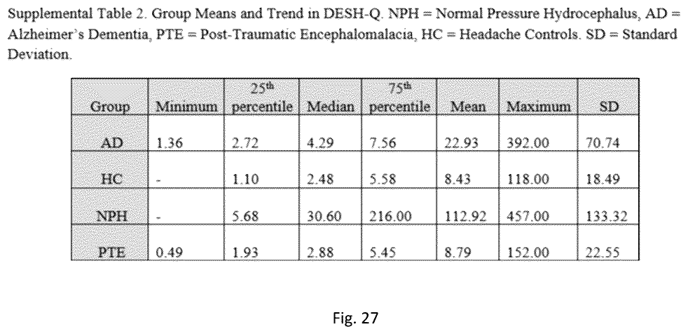

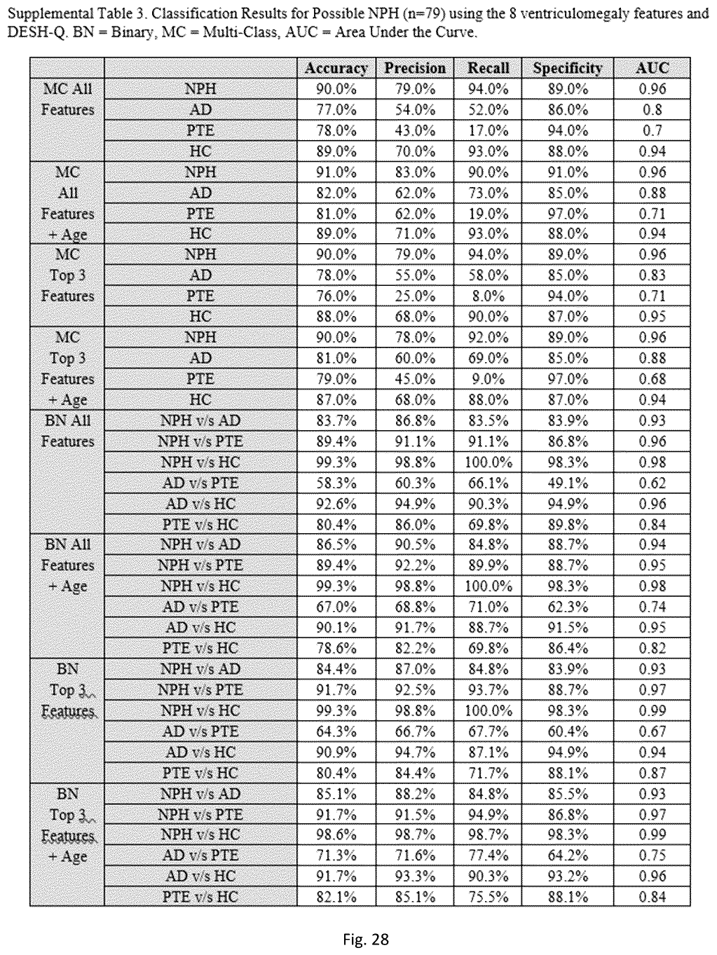

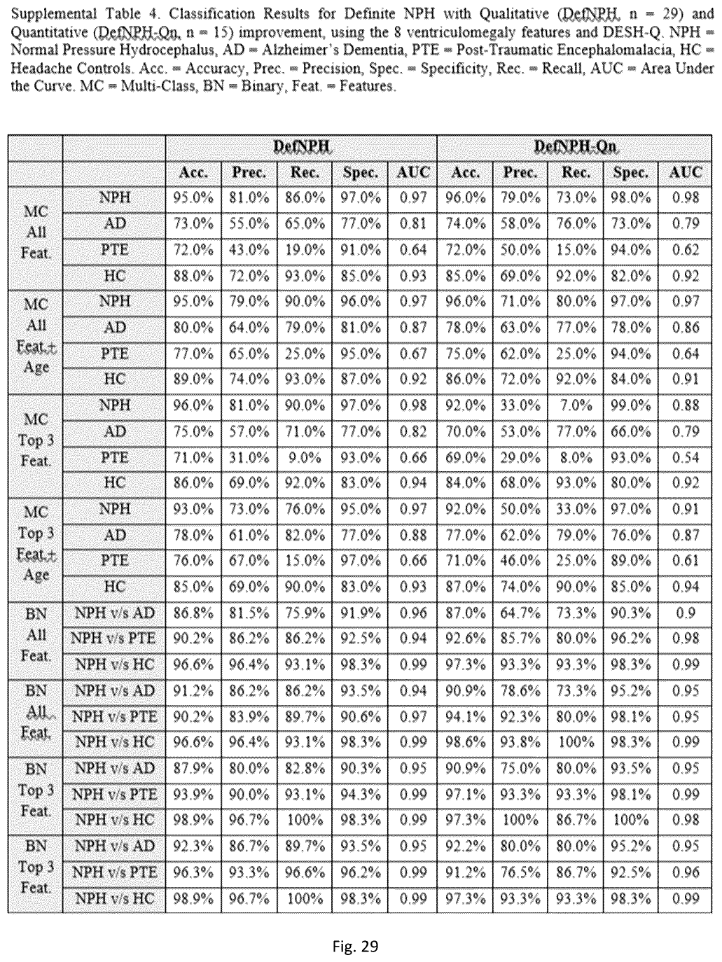

This study was dedicated to the computational assessment of features with inherently objective definitions and can be quantified as ratios of linear measures or angles. As DESH is a prominent descriptive feature of NPH, we examined a computational approach to quantify it using a crude measure DESH-Q, and tested the added benefit it provides to classification. Even though it captured significant differences between NPH and AD/PTE/HC, we found that it had high within-group variation and did not improve the classification performance seen with the eight features of ventriculomegaly presented in this paper. The details of this experiment are presented in the Supplementary Methods and Results (Supplemental FIGS. 23-25, and Supplemental Tables 2-4 shown in FIGS. 27-29). Future works are needed to improve the computational formulation of this prominent feature to produce a robust measure with low variability.

Limitations of this study include the assumption of the HC cohort being “normal” due to a negative head CT. Even though it was confirmed through chart review that these patients did not have a history of dementia, brain injury, or other neurological diseases, undetected pathologies causing headache cannot be completely ruled out. Our definition of patients who improved after shunting in the DefNPH group is a qualitative report as documents of quantitative gait and cognitive tests were not standardized in this retrospective assessment. And there were only 15 of those patients with improvement in quantitative gait measures. In this retrospective design, we could also not assess the distinction between symptomatic NPH patients who improved post-shunting and those who did not, due to a lack of patients in the latter group. A limitation to the generalizability of our work was that more than 90% of the veteran patients involved in this study were male. It needs to be addressed by testing this method on cohorts representing the general population. There were 9 scans where at least one feature was not successfully computed due to severe ventricular occlusion. While they were excluded from this study, feature imputation can be considered in such cases as there were no scans where all features could not be computed.

### Conclusions

We show that (semi) automatically computed features of ventriculomegaly from NCCT can capture different aspects of ventricular morphology in (definite) NPH and successfully distinguish it from HC, and AD/PTE which cause overlapping symptoms like cognitive and gait impairment. We introduced a novel feature called the MaxEccLV that captured significant differences between NPH and the other groups, and a reasonable proxy to the SA which was significantly different between possible NPH and PTE. Following validation (in prospective trials) on larger representative cohorts, this tool has the potential to screen for NPH in clinical or emergency settings that lack advanced imaging and expertise in NPH, to reduce cases of missed and misdiagnoses.

While the methods and systems have been described in connection with specific examples, it is not intended that the scope be limited to the particular embodiments set forth, as the embodiments herein are intended in all respects to be illustrative rather than restrictive. For example, a method for distinguishing central nervous system impairment treatable with shunting (or csf diversion), from that which is untreatable with shunting is contemplated. In some examples, a method for predicting which patients have had a prior brain injury or have impairment resulting in brain imaging metrics not classically associated with normal aging is contemplated. In some examples, a method for classifying the nature of cognitive impairment based on imaging is also contemplated.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
treating a patient diagnosed by a process comprising:
determining, based on a representation of a first tissue, a region of interest of the patient; and
determining, based on the region of interest, an estimate of a location of a commissure within a representation of second tissue of the patient.

2. The method of claim 1, wherein determining the region of interest of the patient comprises:
segmenting, based on the representation of the second tissue of the patient, the first tissue.

3. The method of claim 2, wherein segmenting the first tissue comprises:
isolating, based on a Hounsfield range, the representation of the first tissue; and denoising the representation of the first tissue.

4. The method of claim 1, wherein the estimate of the location of the commissure comprises:
determining, based on the region of interest, a segment of the representation of the second tissue of the patient; and
determining, based on the segment, a heat map of the segment.

5. The method of claim 4, wherein the heat map comprises an intensity indicative of the estimate of the location of the commissure.

6. The method of claim 1, wherein the commissure is a posterior commissure.

7. The method of claim 6, further comprising:
selecting a portion of the representation of the second tissue of the patient; and segmenting, based on the portion of the representation of the second tissue of the patient, a first ventricle of the representation of the second tissue of the patient.

8. The method of claim 7, further comprising: determining, based on the first ventricle, an ellipse; and determining an eccentricity of the ellipse.

9. The method of claim 7, further comprising:
determining a sagittal reconstruction of the representation of the second tissue of the patient;
determining, the representation of the second tissue of the patient and based on a Hounsfield range, slices of the representation of the second tissue of the patient
determining, based on the slices, an axial plane, wherein the first ventricle of the representation of the second tissue of the patient is based on the axial plane.

10. The method of claim 7, further comprising:
determining a second ventricle; and
determining, based on a focal point, a maximum angle between contours of the first ventricle and the second ventricle.

11. The method of claim 7, wherein the first ventricle is a lateral ventricle.

12. The method of claim 1, wherein the commissure is an anterior commissure.

13. The method of claim 1, wherein the first tissue is a ventricle.

14. The method of claim 1, wherein the representation of the second tissue comprises the representation of the first tissue.

15. The method of claim 1, wherein the representation of the second tissue is based on computed tomography scan.

16. The method of claim 7, further comprising:
determining, based on the estimate of the location of the commissure, a condition of the patent, wherein the condition is Normal Pressure Hydrocephalus.

17. The method of claim 16, wherein the condition is determined based on distinguishing the condition from Alzheimer's Dementia, Post-traumatic Encephalomalacia, and headaches.
